(19) United States
(12) Patent Application Publication
(10) Pub. No.: US 2024/0325742 A1
(43) Pub. Date: Oct. 3, 2024

(54) ELECTROPORATION AND ELECTROPHORESIS DEVICES, SYSTEMS, AND METHODS RELATED TO ENDOVASCULAR APPLICATIONS

(71) Applicant: Washington University, St. Louis, MO (US)

(72) Inventors: Mohamed Zayed, St. Louis, MO (US); Guy Genin, St. Louis, MO (US); Christian Zemlin, St. Louis, MO (US); John Cashin, St. Louis, MO (US); Sophia Roberts, St. Louis, MO (US); Devaughn Rucker, St. Louis, MO (US)

(73) Assignee: Washington University, St. Louis, MO (US)

(21) Appl. No.: 18/620,007

(22) Filed: Mar. 28, 2024

Related U.S. Application Data
(60) Provisional application No. 63/492,551, filed on Mar. 28, 2023.

(51) Int. Cl. A61N 1/32; A61N 1/05

(52) U.S. Cl. CPC A61N 1/327; A61N 1/05

(57) ABSTRACT

Among the various aspects of the present disclosure is the provision of an electrophoresis and electroporation device, system, and methods of use thereof. Devices to administer drugs with electrophoresis and electroporation are described. Systems to administer drugs with an electrophoresis and electroporation device by applying voltages with a power source and controller are also described. Also disclosed are methods of administering a therapeutic agent with an electrophoresis and electroporation device or system.

### CROSS-REFERENCE TO RELATED APPLICATIONS

This application claims the benefit of priority to U.S. Provisional Application Ser. No. 63/492,551 filed on Mar. 28, 2023, which is incorporated herein by reference in its entirety.

### STATEMENT REGARDING FEDERALLY SPONSORED RESEARCH OR DEVELOPMENT

This work was supported by the U.S. Department of Veterans Affairs, and the Federal Government has certain rights in this invention.

### MATERIAL INCORPORATED-BY-REFERENCE

Not applicable.

### FIELD OF THE INVENTION

The present disclosure generally relates to electroporation and electrophoresis devices, systems, and methods related to endovascular applications.

### BACKGROUND OF THE INVENTION

Current endovascular delivery of therapeutic agents (pharmacological, genetic, etc.) to the wall of a vessel involves the expansion of an endoluminal angioplasty balloon (FIG. 1B) or deployment of a stent coated with the agent (FIG. 1A). After deflating the angioplasty balloon (FIG. 1C), the agent delivered to the endoluminal surface is gradually washed away from the luminal surface with blood flow. If the agent is embedded in a stent, the delivery of the agent through the endoluminal surface is prolonged. However, this delivery method involves the implantation of permanent or semi-permanent stent material in the vessel lumen with associated risks.

There is a need to develop technology that will allow for more effective delivery of therapeutic agents into the intima and media layers of a blood vessel without the need to leave foreign material within the vessel, and without having the agent of interest wash away with blood flow from the treatment site.

### SUMMARY OF THE INVENTION

Among the various aspects of the present disclosure is the provision of an electrophoresis and electroporation device, system, and methods of use thereof.

Briefly, therefore, the present disclosure is directed to devices and systems for endovascular electrophoresis and electroporation to enhance drug delivery from a drug-eluting balloon.

The present teachings include a device for intraluminal administration of a therapeutic agent within a circulatory vessel. In one aspect, the device can include a first array of positive electrodes. In another aspect, the device can include a second array of negative electrodes. In another aspect, the device can include an amount of a therapeutic agent. In yet another aspect, the device can include an expandable substrate that includes an outer surface. In another aspect, the first array and the second array can be attached to the outer surface of the expandable substrate in an array configuration. In another aspect, the amount of the therapeutic agent can be coated over at least a portion of the outer surface of the expandable substrate. In another aspect, the expandable substrate can be configured to reversibly reposition from a collapsed configuration to an expanded configuration. In another aspect, the collapsed configuration can be configured to facilitate insertion of the device into a circulatory vessel, and the expanded configuration is configured to compress the outer surface against a luminal wall of the circulatory vessel. In another aspect, the first array and second array can be configured to induce at least one electric field within the luminal wall when a voltage is applied between the positive electrodes of the first array and the negative electrodes of the second array when the expandable substrate is positioned in the expanded configuration. In another aspect, the at least one electric field induced by the first and second arrays can be sufficient to cause electroporation of a cellular membrane of the luminal wall. In another aspect, the array configuration can be selected from at least one of a longitudinal pattern, a horizontal pattern, an oblique pattern, a spiral pattern, an auxetic pattern, and any combination thereof. In some embodiments, at least a portion of the first array of positive electrodes, the second array of negative electrodes, or any combination thereof protrudes outward from the outer surface of the expandable substrate. In some embodiments, the outer surface of the expandable substrate can be compliant or non-compliant when the expandable substrate is positioned in the expanded configuration. In yet another aspect, the expandable substrate can be selected from an angioplasty balloon, an expandable stent, and any combination thereof. In some embodiments, the therapeutic agent can be a charged compound and the at least one electric field induced by the first and second arrays can be sufficient to cause electroporation of the cellular membrane of the luminal wall and electrophoresis to facilitate the intraluminal administration of the therapeutic agent. In some embodiments, the therapeutic agent can be an encapsulated agent, wherein the encapsulated agent can be configured to release in response to a release factor selected from stretching, heating, changes to pH, releasing of a cofactor, photoactivation, irradiation, application of electrical fields of variable strength and frequency, and any combination thereof. In one aspect, the device can include a power source operatively coupled to the first array of positive electrodes and to the second array of negative electrodes, and a controller operatively coupled to the power source. In one aspect, the power source can be configured to apply the voltage between the positive electrodes of the first array and the negative electrodes of the second array. In another aspect, the controller can be configured to operate the power source at a predetermined variable strength and frequency, the predetermined strength and frequency configured to induce electroporation, electrophoresis, or any combination within the luminal wall of the circulatory vessel.

The present teachings also include a system for intraluminal administration of a therapeutic agent within a circulatory vessel. In one aspect, the system can include the device described above, a power source operatively coupled to the first array of positive electrodes and to the second array of negative electrodes, and a controller operatively coupled to the power source. In one aspect, the power source can be configured to apply a voltage between the positive electrodes of the first array and the negative electrodes of the second array. In another aspect, the controller can be configured to operate the power source at a predetermined variable voltage strength and frequency, the predetermined variable strength and frequency configured to induce electroporation, electrophoresis, or any combination thereof within the luminal wall of the circulatory vessel.

The present teachings also include a method for intraluminal administration of a therapeutic agent within a circulatory vessel. In one aspect, the method can include providing the device or system described above, positioning the expandable substrate in the collapsed configuration within a lumen of the circulatory vessel, repositioning the expandable substrate into the expanded configuration to press the positive electrodes of the first array, the negative electrodes of the second array, and the amount of the therapeutic agent against the luminal lining of the circulatory vessel, operating the power source to apply a voltage to the positive electrodes of the first array and the negative electrodes of the second array and to induce at least one electric field within the luminal wall, the at least one electric field configured to induce electroporation, electrophoresis, or any combination thereof within the luminal wall of the circulatory vessel, wherein the induced electroporation, electrophoresis, or any combination thereof facilitate the intraluminal administration of the therapeutic agent. In another aspect, the method can include repositioning the expandable substrate into the collapsed configuration, and removing the expandable substrate in the collapsed configuration from within the lumen of the circulatory vessel.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is an illustration of an initial phase of an existing method of endovascular application of agents to a blood vessel endoluminal surface, showing a catheter-mounted deflated balloon coated with the agent to be applied that is advanced onto a guidewire in a collapsed configuration.

FIG. 1B shows an illustration of a subsequent phase of the existing method illustrated in FIG. 1A, in which the angioplasty balloon is inflated to press the agent against the endoluminal surface.

FIG. 1C shows an illustration of a subsequent phase of the existing method illustrated in FIGS. 1A and 1B, in which the balloon is deflated, and the agent theoretically remains on the endoluminal surface before it is washed away downstream with blood flow.

FIGS. 2A, 2B, 2C, and 2D contain a series of schematics depicting the employment of a device as described herein that includes balloon-mounted electrodes, and its method of employment in the endovascular application of agents to the blood vessel endoluminal surface.

FIG. 2A shows an initial phase in which the catheter-mounted deflated balloon in a deflated configuration with the agent to be applied above and around the electrodes on its surface (black) is advanced within an endovascular lumen onto a guidewire.

FIG. 2B shows a phase subsequent to the initial phase illustrated in FIG. 2A, in which the balloon is inflated to contact the endoluminal surface.

FIG. 2C shows a phase subsequent to the phase illustrated in FIG. 2B, in which an electric voltage is applied between the electrodes to electroporate the endoluminal surface and electrophorate the charged agent beyond the endoluminal surface and into the intraluminal wall of the vessel.

FIG. 2D shows a phase subsequent to the phase illustrated in FIG. 2B, in which the applied voltage is terminated and the electrode-coated balloon is deflated and retracted, leaving the agent embedded in the vessel wall where it can have a more lasting impact.

FIGS. 3A, 3B, and 3C contain schematic illustrations of various embodiments of electrode orientations of the device disclosed herein. The electrodes may be oriented on the surface of the angioplasty balloon in many different configurations depending on utility, application, and versatility.

FIG. 3A is a schematic illustration of a device with an electrode array in which the electrodes consist of wires that do not significantly alter the smooth surface of the balloons, in which each electrode is positioned in a circumferential orientation relative to the balloon; a voltage is applied in an alternating positive/negative voltage pattern over the series of circumferential electrodes distributed along the length of the balloon.

FIG. 3B is a schematic illustration of the device of FIG. 3A, in which a positive voltage is applied to a portion of the circumferential electrodes over one half of the balloon, and a negative voltage is applied to a portion of the circumferential electrodes over the other half of the balloon.

FIG. 3C is a schematic illustration of an electrode array wherein the electrodes consist of wires that do not significantly alter the smooth surface of the balloons, in which the electrodes are oriented in a spiral path along the length of the balloon and the voltage is applied to the spiral electrodes in an alternating positive/negative voltage pattern.

FIG. 3D is a schematic illustration of an electrode array wherein the electrodes consist of wires that do not significantly alter the smooth surface of the balloons, in which the electrodes are oriented in a longitudinal direction along the length of the balloon and the voltage is applied to the longitudinal electrodes in an alternating positive/negative voltage pattern.

FIG. 3E is a schematic of an electrode array wherein electrodes protrude from the surface of the balloon, adding bumps and ridges for further electroporation impact on the vessel wall, in which the electrodes are arranged in circumferential groups, in which each member of a circumferential group is aligned longitudinally with corresponding electrode from adjacent circumferential groups. A voltage is applied to circumferential groups of electrodes in an alternating positive/negative pattern along the length of the balloon.

FIG. 3F is a schematic illustration of the device of FIG. 3E in which the voltage is applied to longitudinal groups of electrodes in an alternating positive/negative pattern around the circumference of the balloon.

FIG. 4 is a schematic illustration of a device with an electrode array arranged in an auxetic material pattern typically seen for wires in expandable endovascular stents. Without being limited to any particular theory, such stents have patterns of wires that enable them to expand to many times their diameter without straining the wires forming the endoskeleton of the stent.

FIG. 5 is a schematic illustration of a device that includes protruding circumferential electroporation electrodes arranged along the length of the device. To make electroporation effective, it is particularly desirable to have the electrodes press into the tissue in ridges. This arrangement of electrodes, and the resulting electroporation of the vessel wall, facilitate the transfer of agents into the vessel wall media and intima.

FIG. 6A contains a DAPI and PI image (left) and graph (right) showing PI uptake by a VMSC monotayer after electroporation and electrophoresis at a voltage of 0 V.

FIG. 6B contains a DAPI and PI image (left) and graph (right) showing PI uptake by a VMSC monotayer after electroporation and electrophoresis at a voltage of 160 V . . .

FIG. 6C contains a DAPI and PI image (left) and graph (right) showing PI uptake by a VMSC monotayer after electroporation and electrophoresis at a voltage of 320 V.

FIG. 6D contains a DAPI and PI image (left) and graph (right) showing PI uptake by a VMSC monotayer after electroporation and electrophoresis at a voltage of 480 V.

FIG. 6E contains a DAPI and PI image (left) and graph (right) showing PI uptake by a VMSC monotayer after electroporation and electrophoresis at a voltage of 640 V.

FIG. 7A contains a DAPI and PI image showing PI uptake by a pig aortic segment after electroporation and electrophoresis at a voltage of 0 V.

FIG. 7B contains a DAPI and PI image showing PI uptake by a pig aortic segment after electroporation and electrophoresis at a voltage of 160 V.

FIG. 7C contains a DAPI and PI image showing PI uptake by a pig aortic segment after electroporation and electrophoresis at a voltage of 320 V.

FIG. 7D is a bar graph summarizing PI uptake by a pig aortic segment after electroporation and electrophoresis based on the DAPI and PI images of FIGS. 7A, 7B, and 7C.

### DETAILED DESCRIPTION OF THE INVENTION

The present disclosure is based, at least in part, on the discovery that an endovascular electrode-coated balloon, stent, or endovascular implant can enhance drug delivery from the endoluminal device using electroporation and electrophoresis. A balloon, stent, or endovascular implant would be attached to a catheter system that can relay electrical pulses to the vascular and organ tissue. As shown herein, devices, systems, and methods related to endovascular applications are described.

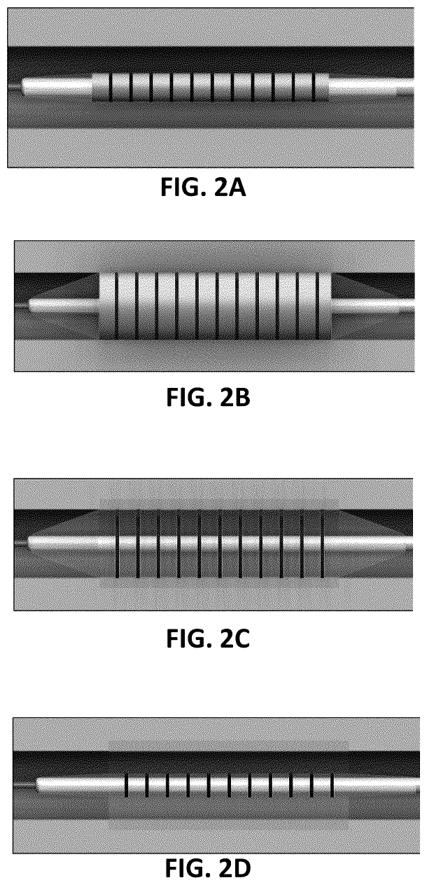

One aspect of the present disclosure provides for endovascular electrode-coated balloon devices, systems, and methods of use thereof. The present disclosure utilizes electroporation and/or electrophoresis to facilitate the endovascular application and intraluminal penetration of therapeutic agents (FIG. 2A, FIG. 2B, FIG. 2C, FIG. 2D).

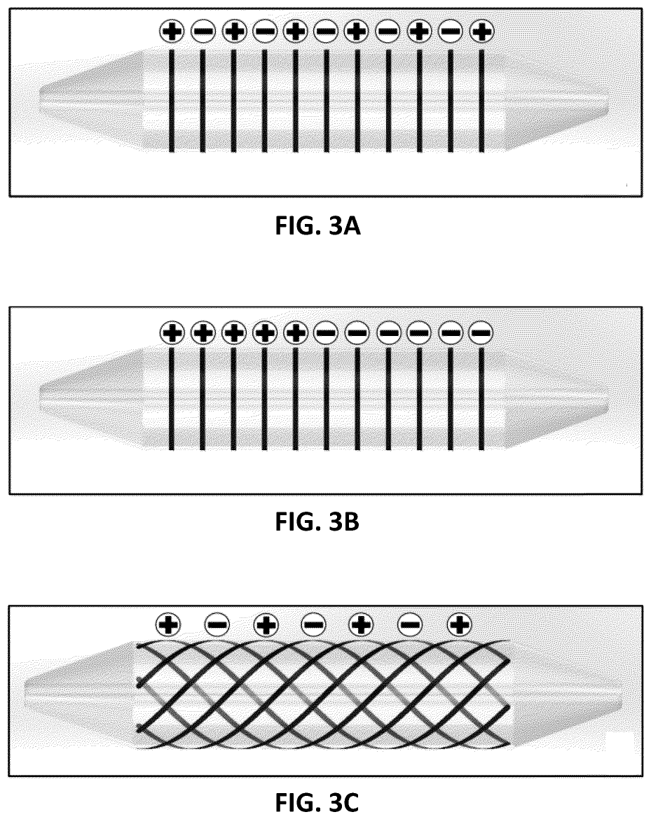

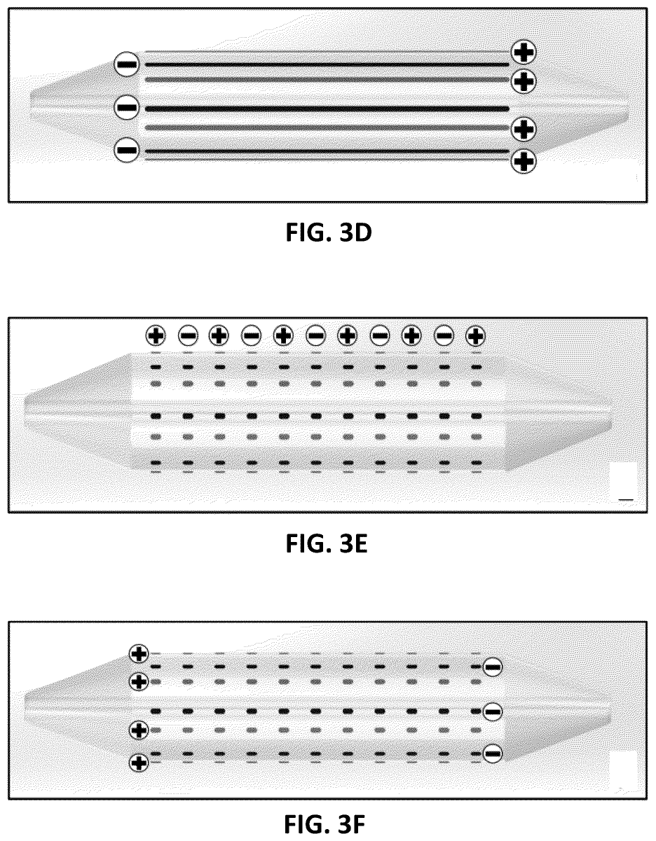

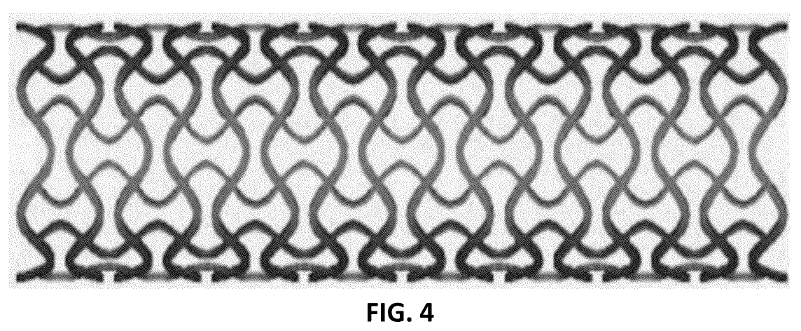

Electrodes may be attached and oriented across the surface of a compliant or non-compliant expandable angioplasty balloon, stent, or other endovascular implants in a variety of different configurations (FIG. 3A, FIG. 3B, FIG. 3C, FIG. 3D, FIG. 3E, FIG. 3F). The electrodes can be composed, but not limited to, a material that can conduct electrical pulses. Electrode configurations can be placed longitudinally, horizontally, oblique, spiral, or in auxetic patterns typically seen for wires in expandable vascular stents (FIG. 4).

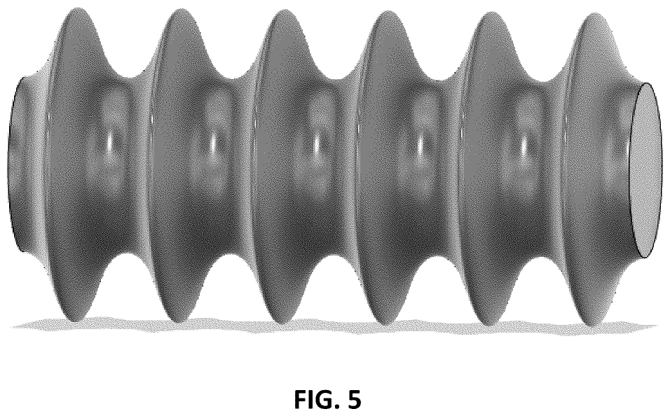

Application of a voltage, with sufficient strength, between positive and negative electrodes exposes the luminal wall to electric fields that results in electroporation of the cellular membranes of the inner vessel wall. Voltage, as defined herein, refers to the difference in electric potential between the positive electrodes (higher electric potential) and the negative electrode (lower electric potential). In some aspects, the lower electric potential of the negative electrode is selected from a zero or grounded electric potential or a negative electric potential. Additionally, in some configurations, the angioplasty balloon can have external electrode protrusions (FIG. 5) for higher penetration of the voltage fields into the vessel wall for effective electroporation and electrophoresis.

Without being limited to any particular theory, the size and strength of electrical fields induced in the luminal wall of the blood vessel depend on any one or more of a plurality of factors including, but not limited to, the applied voltage strength, the spatial and temporal patterns of the voltage applied to the intraluminal surface via the positive and negative electrodes, the degree of protrusion of electrodes from the outer surface of the expandable substrate of the device, the contact area of individual positive electrodes on the intraluminal surface, the spatial separation and arrangement of individual positive and negative electrodes, and any other suitable factor without limitation.

In various aspects, the spatial pattern of the voltage applied to the intraluminal surface is influenced by the structure and arrangement of positive and negative electrodes over the outer surface of the expandable substrate of the device, such as a balloon. In some aspects, the applied voltage may be applied in an alternating pattern between adjacent positive and negative electrodes, as illustrated in FIGS. 3A, 3C, and 3D. In other aspects, the applied voltage may be applied to contiguous groups of electrodes, as illustrated in FIG. 3B. In other additional aspects, the applied voltage may be applied to groups of electrodes that are contiguous along one direction, and in an alternating manner along a second direction. By way of non-limiting example, the same voltage may be applied to contiguous circumferential groups of electrodes, but in an alternating pattern between adjacent circumferential groups of electrodes along the length of the device, as illustrated in FIG. 3E. By way of another non-limiting example, the same voltage may be applied to contiguous groups of electrodes arranged along a longitudinal row, but in an alternating pattern between adjacent longitudinal rows of electrodes with respect to the circumference of the device, as illustrated in FIG. 3F.

In various aspects, the voltage (ranging from 25-3000 V) applied to the intraluminal surface is influenced by the magnitude and temporal pattern of voltage supplied to the arrays of positive and negative electrodes by an operatively coupled power source. In various aspects, the applied voltage may vary in magnitude, frequency, waveform, duration of the application, and any other relevant parameter characterizing a temporal voltage pattern. Without being limited to any particular theory, the peak magnitude and temporal pattern of the applied voltage influence the degree and efficacy of electroporation of the intraluminal surface and/or the efficacy of electrophoresis of therapeutic agents into the luminal layers. In various aspects, the applied voltage may be applied within any known physiologically relevant range in a constant (time-invariant) or time-varying manner without limitation. In some aspects, the voltage may be applied to the arrays of positive and negative electrodes in a time-varying pattern characterized by a frequency and a voltage waveform. Non-limiting examples of suitable voltage waveforms include voltage steps or square-wave waveforms, sinusoidal waveforms, sawtooth waveforms, and any other suitable waveform without limitation.

In some aspects, the therapeutic agents coating the surface of the angioplasty balloon or other expandable substrate may be charged, allowing for electrophoresis of the agent in addition to electroporation of the intraluminal surface. Reversible and irreversible electroporation and electrophoresis may be applied using different electrode arrays and configurations and different applied voltage patterns as described above. The device may additionally contain an encapsulation of the therapeutic agent that is configured to release upon exposure to a releasing stimulus or signal. Non-limiting examples of suitable releasing stimuli or signals include stretch, heating, changes to pH, release of a cofactor, photoactivation, irradiation, application of electrical fields of variable strength and frequency, and any combination thereof.

A potential application of intraluminal electroporation and/or electrophoresis can be the application of therapeutics to reduce the risk of intimal hyperplasia. With electroporation and/or electrophoresis permeability to the arterial medial layer and smooth muscle cells can be increased. This allows the therapeutics to effectively enter into the target tissue and reduce smooth muscle cell migration. Reduced smooth muscle cell migration leads to reduced intimal hyperplasia and improved arterial patency post-endovascular intervention.

After electroporation, with and without electrophoresis, the balloon or other expandable substrate with its overlying attached electrode arrays can be deflated and removed from the vessel lumen, leaving behind the agent well integrated into the vessel lumen wall.

### Cardiovascular Modulation Agents

As described herein, in some aspects, therapeutic agents to treat cardiovascular disease or cardiovascular modulation agents can be coated onto the surface of the balloon device of the current disclosure and released through electroporation and electrophoresis by applying a voltage to the balloon-mounted electrodes.

Expression of various pathways has been implicated in various diseases, disorders, and conditions, including cardiovascular disease. As such, modulation of gene expression or other biological machinery associated with cardiovascular disease can be used for the treatment of such conditions. A cardiovascular modulation agent can modulate cardiovascular response or induce or inhibit cardiovascular disease. Cardiovascular modulation can comprise modulating the expression of genes associated with cardiovascular disease on cells, modulating the quantity of cells that express genes associated with cardiovascular disease, or modulating the quality of the genes associated with cardiovascular disease in or on cells.

Cardiovascular modulation agents can be any composition or method that can modulate gene expression on cells associated with cardiovascular disease (e.g., matrix metalloproteinases (MMP) and G-protein-coupled receptors (GPCR)). For example, a cardiovascular modulation agent can be an activator, an inhibitor, an agonist, or an antagonist. As another example, the cardiovascular modulation can be the result of gene editing.

A cardiovascular modulation agent can be an antibody (e.g., a monoclonal antibody to a GPCR associated with cardiovascular disease).

A cardiovascular modulating agent can be an agent that induces or inhibits progenitor cell differentiation into cardiovascular disease gene-expressing cells (e.g., cells expressing an MMP and/or GPRC associated with cardiovascular disease). For example, ACE inhibitors such as ramipil can be used to block ACE receptors. As another example, angiotensin-II antagonists such as losartan can be used to block angiotensin-II. In yet another example, beta-blockers such as bisoprolol can be used to block beta receptors. As another example, smooth muscle cell migration inhibitors such as paclitaxel or sirolimus can be used to block cell migration and intimal hyperplasia.

Cardiovascular Disease Signal Reduction, Elimination, or Inhibition by Small Molecule Inhibitors, shRNA, siRNA, or ASOs

As described herein, a cardiovascular modulation agent can be used for use in therapy for cardiovascular disease. A cardiovascular modulation agent can be used to reduce/eliminate or enhance/increase signals associated with cardiovascular health and disease. For example, a cardiovascular modulation agent can be a small molecule inhibitor of angiotensin-converting enzyme (ACE) or angiotensin-II. As another example, a cardiovascular modulation agent can be a short hairpin RNA (shRNA). As another example, a cardiovascular modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### Cardiovascular Disease Inhibiting Agent

One aspect of the present disclosure provides for targeting of ACE, its receptor, or its downstream signaling. The present disclosure provides methods of treating or preventing cardiovascular disease based on the discovery that delivery of a drug for cardiovascular disease from a drug-eluting endovascular balloon can improve outcomes, and that balloon-mounted electrodes can enhance delivery using electrophoresis and electroporation.

As described herein, inhibitors of ACE and other factors associated with cardiovascular disease (e.g., antibodies, fusion proteins, small molecules) can reduce or prevent cardiovascular disease. A cardiovascular disease inhibiting agent can be any agent that can inhibit signals associated with cardiovascular disease, downregulate signals associated with cardiovascular disease, or knockdown signals associated with cardiovascular disease.

As an example, a cardiovascular disease inhibiting agent can inhibit signaling associated with cardiovascular disease.

For example, the cardiovascular disease inhibiting agent can be an anti-ACE antibody. Furthermore, the anti-ACE antibody can be a murine antibody, a humanized murine antibody, or a human antibody.

As another example, the cardiovascular disease inhibiting agent can be an anti-angiotensin-II antibody, wherein the anti-angiotensin-II antibody prevents the binding of angiotensin-II to its receptor or prevents activation of angiotensin-II and downstream signaling.

As another example, the cardiovascular disease inhibiting agent can be a fusion protein. For example, the fusion protein can be a decoy receptor for ACE. Furthermore, the fusion protein can comprise a mouse or human Fc antibody domain fused to the ectodomain of ACE or angiotensin-II.

As another example, a cardiovascular disease inhibiting agent can be ramipril, which has been shown to be a potent and specific inhibitor of ACE signaling.

As another example, a cardiovascular disease inhibiting agent can be an inhibitory protein that antagonizes ACE, angiotensin, or beta receptors. For example, the cardiovascular disease inhibiting agent can be a viral protein, which has been shown to antagonize genes and their products that are associated with cardiovascular disease.

As another example, a cardiovascular disease inhibiting agent can be a short hairpin RNA (shRNA) or a short interfering RNA (siRNA) targeting ACE, angiotensin-II, or beta receptors.

As another example, a cardiovascular disease inhibiting agent can be a sgRNA targeting ACE, angiotensin-II, or beta receptors.

Methods for preparing a cardiovascular disease inhibiting agent (e.g., an agent capable of inhibiting signaling associated with cardiovascular disease) can comprise the construction of a protein/Ab scaffold containing the natural angiotensin-II receptor as an angiotensin-II neutralizing agent; developing inhibitors of the angiotensin-II receptor “down-stream”; or developing inhibitors of angiotensin-II production “up-stream”.

Inhibiting ACE, angiotensin-II, or beta receptors can be performed by genetically modifying the genes in a subject or genetically modifying a subject to reduce or prevent expression of the genes associated with cardiovascular disease, such as through the use of CRISPR-Cas9 or analogous technologies, wherein, such modification reduces or prevents cardiovascular disease.

### Chemical Agent:

Examples of therapeutic agents for cardiovascular disease are described herein.

R groups can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include-OH. The “hydroxyl” can be optionally substituted.

The terms “halogen” and “halo”, as used herein, unless otherwise indicated, include chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, includes a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C1-10 alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH2-cyclopropyl, —O—CH2-cyclobutyl, —O—CH2-cyclopentyl, —O—CH2-cyclohexyl, —O—CH2-cycloheptyl, —O—CH2-cyclooctyl, —O—CH2-cyclononyl, —O—CH2-cyclodecyl, —O—(CH2) 2-cyclopropyl, —O—(CH2) 2-cyclobutyl, —O—(CH2) 2-cyclopentyl, —O—(CH2) 2-cyclohexyl, —O—(CH2) 2-cycloheptyl, —O—(CH2) 2-cyclooctyl, —O—(CH2) 2-cyclononyl, or —O—(CH2) 2-cyclodecyl. An alkoxyl can be saturated, partially saturated, or unsaturated. The “alkoxyl” can be optionally substituted.

The term “cycloalkyl”, as used herein, unless otherwise indicated, can include an aromatic, non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C3-10 cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include-lower alkyl-cycloalkyl, wherein lower alkyl and cycloalkyl are as defined herein. Examples of -lower alkyl-cycloalkyl groups include, but are not limited to, —CH2-cyclopropyl, —CH2-cyclobutyl, —CH2-cyclopentyl, —CH2-cyclopentadienyl, —CH2-cyclohexyl, —CH2-cycloheptyl, or —CH2-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “hetreocyclic” can be optionally substituted.

The term “indole”, as used herein, is an aromatic heterocyclic organic compound with the formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatography. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, which further can include a stoichiometric or non-stoichiometric amount of water bound by non-covalent intermolecular forces.

In some embodiments, the therapeutic agent for cardiovascular disease can be, but is not limited to, Ramipril, losartan, amiodarone, warfarin, aspirin, bisoprolol, and amlodipine.

### Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing a cardiovascular disease in a subject in need of administration of a therapeutically effective amount of a cardiovascular therapeutic agent, so as to treat cardiovascular disease.

Methods described herein are generally performed on a subject in need thereof. A subject in need of the therapeutic methods described herein can be a subject having, diagnosed with, suspected of having, or at risk for developing cardiovascular disease. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a cardiovascular therapeutic agent is, for example, an amount that would cause the desired therapeutic effect in a subject while minimizing undesired side effects. In various embodiments, an effective amount of a cardiovascular therapeutic agent described herein can substantially inhibit cardiovascular disease, slow the progress of the cardiovascular disease, or limit the development of cardiovascular disease.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration. In an exemplary example, administration occurs by coating the therapeutic agent on the balloon device described in the present disclosure, inserting the balloon endovascularly into a subject, and applying a voltage to the balloon-mounted electrodes to cause electrophoresis and electroporation.

When used in the treatments described herein, a therapeutically effective amount of a cardiovascular therapeutic can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat cardiovascular disease.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a cardiovascular therapeutic agent can occur as a single event or over a time course of treatment. For example, a cardiovascular therapeutic agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for cardiovascular disease.

A cardiovascular therapeutic agent can be administered simultaneously or sequentially with another agent, such as an antibiotic, an anti-inflammatory, or another agent. For example, a cardiovascular therapeutic agent can be administered simultaneously with another agent, such as an antibiotic or an anti-inflammatory. Simultaneous administration can occur through the administration of separate compositions, each containing one or more of a cardiovascular therapeutic agent, an antibiotic, an anti-inflammatory, or another agent. Simultaneous administration can occur through the administration of one composition containing two or more of a cardiovascular therapeutic agent, an antibiotic, an anti-inflammatory, or another agent. A cardiovascular therapeutic agent can be administered sequentially with an antibiotic, an anti-inflammatory, or another agent. For example, a cardiovascular therapeutic agent can be administered before or after the administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

In some embodiments, a therapeutic agent can be coated onto the balloon device described in the present disclosure. In some embodiments, more than one cardiovascular therapeutic agent can be coated on the balloon device. In some embodiments, more than one of a cardiovascular therapeutic agent, an antibiotic, an anti-inflammatory, or another agent can be coated on the balloon device. As such, administration can be performed by inserting the drug-coated balloon endovascularly into a subject and applying a voltage to the balloon-mounted electrodes to cause electrophoresis and/or electroporation.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to the balloon device described in the present disclosure, sterilizing materials, and other surgical instruments needed for endovascular surgery. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1-Endovascular Application of Electroporation and Electrophoresis

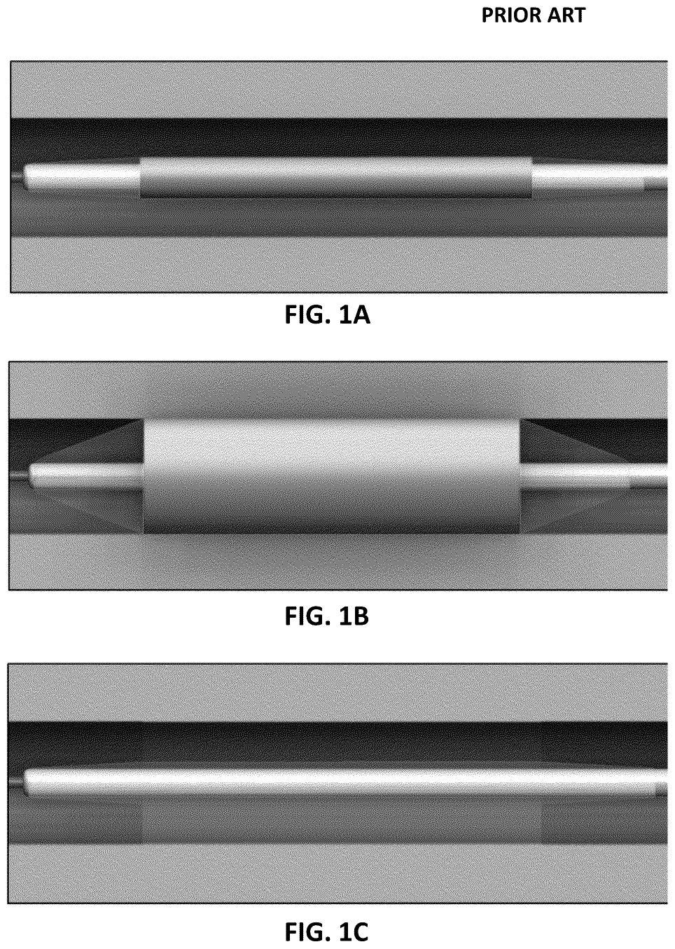

In this example, an endovascular drug-coated balloon device with electrode attachments is described to enhance drug delivery. Employment of the device can be seen in FIG. 2, which improves on the current standard of care (FIG. 1) wherein the catheter-mounted deflated balloon with the agent to be applied above and around the electrodes on its surface (black) is advanced endovascularly on to a guidewire (FIG. 2A). The balloon is inflated to contact the endoluminal surface (FIG. 2B). Electric voltage is applied between the electrodes to electroporate the endoluminal surface and electrophorate the charged agent beyond the endoluminal surface and into intraluminal wall of the vessel (FIG. 2C). The voltage is eliminated and the electrode-coated balloon is deflated and retracted, leaving the agent imbedded in the vessel wall where it can have a more lasting impact (FIG. 2D). The electrodes can be in placed in several different orientations, and can be wires that do not significantly alter the smooth surface of the balloon (FIG. 3A, FIG. 3B, FIG. 3C, FIG. 3D) or may protrude from the surface of the balloon, adding bumps and ridges for further electroporation impact on the vessel wall (FIG. 3E, FIG. 3F). An example of auxetic material patterns typically seen for wires in expandable endovascular stents can be seen in FIG. 4, and an example schematic of protruding electroporation electrodes can be seen in FIG. 5.

### Example 2-Delivery of Propidium Iodide Using Endovascular Electroporation and Electrophoresis

To demonstrate the delivery of a therapeutic agent using electroporation and electrophoresis similar to that used by the disclosed endovascular devices, the following experiments were conducted.

Human vascular smooth muscle cells (VSMCs) were plated at passage 7 on fibrin-coated 2-well chamber slides. Propidium Iodide (PI) at a concentration of 1 mg/mL was added to each slide, and tungsten pin electrodes were brought into contact with the VMSC monolayers in each 2-well chamber slide without pressure. Electroporation and electrophoresis were applied to the VMSC monolayers via the tungsten pin electrodes using applied voltage levels of 0V, 160V, 320V, 480V, and 640V. After treatment, the VMSC monolayers were fixed in PFA and imaged for DAPI and PI.

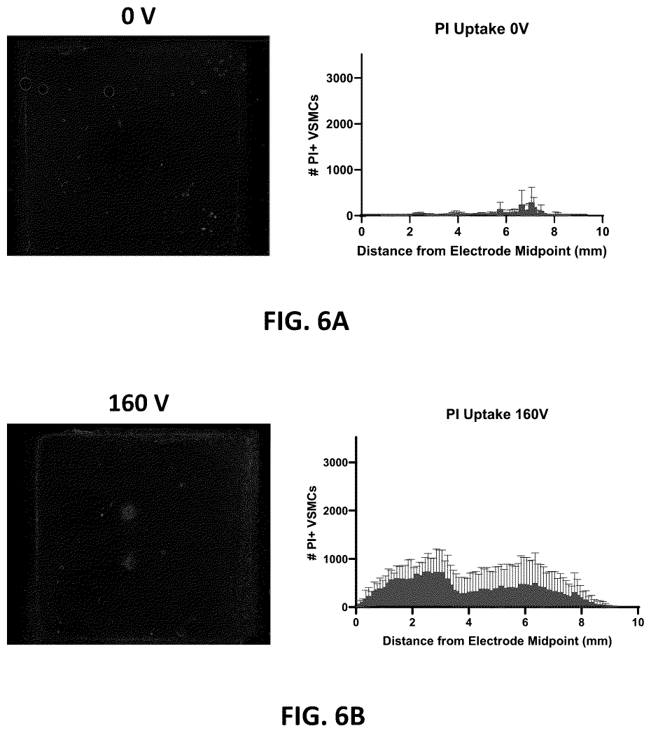

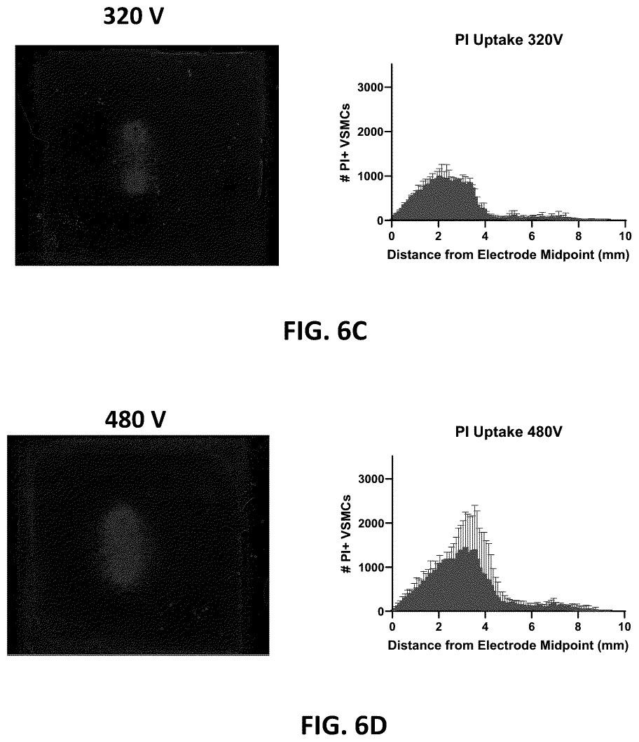

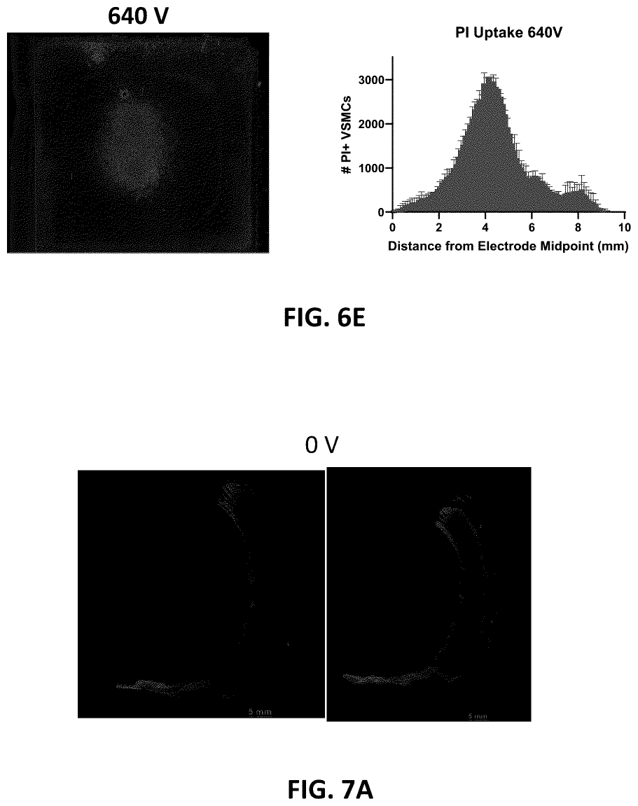

The DPAI and PI images (left) and estimated PI uptake of the VMSC monolayers are presented for applied voltage levels of 0V (FIG. 6A), 160V (FIG. 6B), 320V (FIG. 6C), 480V (FIG. 6D), and 640V (FIG. 6E). Electroporation and electrophoresis resulted in PI uptake by the VMSC monolayers in a voltage-dependent manner.

The results of these experiments validated the capability of electroporation and electrophoresis for the delivery of therapeutic compounds to VMSCs.

### Example 3-Endovascular Delivery of Propidium Iodide Using Endovascular Electroporation and Electrophoresis

To demonstrate the endovascular delivery of a therapeutic agent using electroporation and electrophoresis similar to that used by the disclosed endovascular devices, the following experiments were conducted.

Pig aortic segments were mounted in a preparation and contacted with propidium Iodide (PI) at a concentration of 1 mg/mL. Tungsten pin electrodes were brought into contact with the aortic segment preparations. Electroporation and electrophoresis were applied to the aortic segments via the tungsten pin electrodes using applied voltage levels of 0V, 160V, and 320V. After treatment, the aortic layers were fixed in PFA and imaged for DAPI and PI.

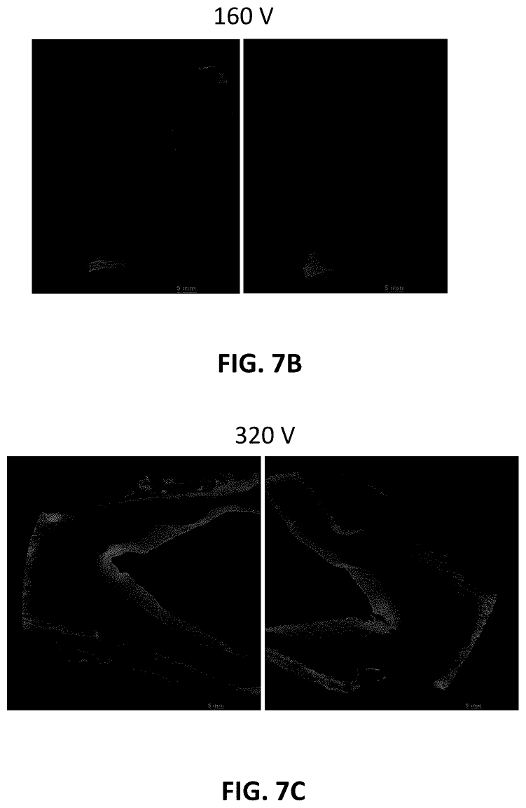

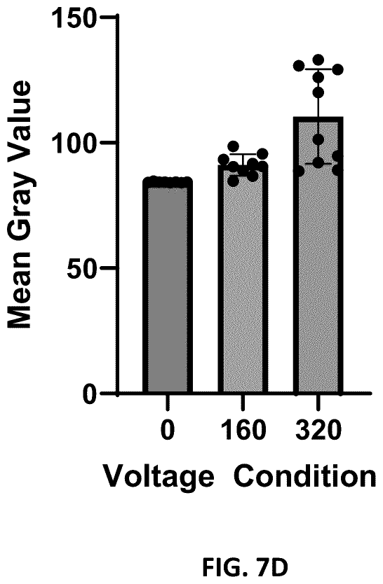

The PI resulting images are presented for applied voltage levels of 0V (FIG. 7A), 160V (FIG. 7B), and 320V (FIG. 7C). FIG.>7D is a bar graph summarizing the PI uptakes estimated from the PI images of FIGS. 7A, 7B, and 7C. Electroporation and electrophoresis resulted in PI uptake by the pig aortic segments in a voltage-dependent manner.

The results of these experiments validated the capability of electroporation and electrophoresis for the endovascular delivery of therapeutic compounds.

What is claimed is:

1. A device for intraluminal administration of a therapeutic agent within a circulatory vessel, the device comprising a first array of positive electrodes, a second array of negative electrodes, an amount of a therapeutic agent, and an expandable substrate comprising an outer surface, wherein:
a. the first array and the second array are attached to the outer surface of the expandable substrate in an array configuration;
b. the amount of the therapeutic agent is coated over at least a portion of the outer surface of the expandable substrate;
c. the expandable substrate is configured to reversibly reposition from a collapsed configuration to an expanded configuration, wherein the collapsed configuration is configured to facilitate insertion of the device into a circulatory vessel, and the expanded configuration is configured to compress the outer surface against a luminal wall of the circulatory vessel; and
d. the first array and second array are configured to induce at least one electric field within the luminal wall when a voltage is applied between the positive electrodes of the first array and the negative electrodes of the second array when the expandable substrate is positioned in the expanded configuration.

2. The device of claim 1, wherein the at least one electric field induced by the first and second arrays is sufficient to cause electroporation of a cellular membrane of the luminal wall.

3. The device of claim 1, wherein the array configuration is selected from at least one of a longitudinal pattern, a horizontal pattern, an oblique pattern, a spiral pattern, an auxetic pattern, and any combination thereof.

4. The device of claim 1, wherein at least a portion of the first array of positive electrodes, the second array of negative electrodes, or any combination thereof protrudes outward from the outer surface of the expandable substrate.

5. The device of claim 1, wherein the outer surface of the expandable substrate is compliant or non-compliant when the expandable substrate is positioned in the expanded configuration.

6. The device of claim 1, wherein the expandable substrate is selected from an angioplasty balloon, an expandable stent, and any combination thereof.

7. The device of claim 1, wherein the therapeutic agent comprises a charged compound and the at least one electric field induced by the first and second arrays is sufficient to cause electroporation of the cellular membrane of the luminal wall and electrophoresis to facilitate the intraluminal administration of the therapeutic agent.

8. The device of claim 1, wherein the therapeutic agent is an encapsulated agent, wherein the encapsulated agent is configured to release in response to a release factor selected from stretching, heating, changes to pH, releasing of a cofactor, photoactivation, irradiation, application of electrical fields of variable strength and frequency, and any combination thereof.

9. The device of claim 1, further comprising a power source operatively coupled to the first array of positive electrodes and to the second array of negative electrodes, and a controller operatively coupled to the power source, wherein:
a. the power source is configured to apply the voltage between the positive electrodes of the first array and the negative electrodes of the second array; and
b. the controller is configured to operate the power source at a predetermined variable strength and frequency, the predetermined strength and frequency configured to induce electroporation, electrophoresis, or any combination within the luminal wall of the circulatory vessel.

10. A system for intraluminal administration of a therapeutic agent within a circulatory vessel, comprising the device of claim 1, a power source operatively coupled to the first array of positive electrodes and to the second array of negative electrodes, and a controller operatively coupled to the power source, wherein:
a. the power source is configured to apply a voltage between the positive electrodes of the first array and the negative electrodes of the second array; and
b. the controller is configured to operate the power source at a predetermined variable voltage strength and frequency, the predetermined variable strength and frequency configured to induce electroporation, electrophoresis, or any combination thereof within the luminal wall of the circulatory vessel.

11. A method for intraluminal administration of a therapeutic agent within a circulatory vessel, the method comprising:
a. providing the device or system of claim 1;
b. positioning the expandable substrate in the collapsed configuration within a lumen of the circulatory vessel;
c. repositioning the expandable substrate into the expanded configuration to press the positive electrodes of the first array, the negative electrodes of the second array, and the amount of the therapeutic agent against the luminal lining of the circulatory vessel; and
d. operating the power source to apply a voltage to the positive electrodes of the first array and the negative electrodes of the second array and to induce at least one electric field within the luminal wall, the at least one electric field configured to induce electroporation, electrophoresis, or any combination thereof within the luminal wall of the circulatory vessel, wherein the induced electroporation, electrophoresis, or any combination thereof facilitate the intraluminal administration of the therapeutic agent.

12. The method of claim 11, further comprising:
a. repositioning the expandable substrate into the collapsed configuration; and
b. removing the expandable substrate in the collapsed configuration from within the lumen of the circulatory vessel.
